(19) United States
(12) Patent Application Publication
(10) Pub. No.: US 2008/0234800 A1
(43) Pub. Date: Sep. 25, 2008

(54) Stent Including a Toggle Lock

(75) Inventor: Gerry Clarke, Moycullen (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/688,446

(22) Filed: Mar. 20, 2007

(51) Int. Cl. A61F 2/06

(52) U.S. Cl. 623/1.16

(57) ABSTRACT

A stent includes cylindrical rings disposed adjacent to each other. Each cylindrical ring includes longitudinal segments that may be disposed generally parallel to a longitudinal axis of the stent. The longitudinal segments are coupled to each other by toggle lock struts. The toggle lock struts are disposed substantially at the center of the circumferentially adjacent longitudinal segments. When the stent is in a compressed state for delivery, the toggle lock struts are bent such that the longitudinal segments are disposed close to each other. Upon expansion of the stent, the toggle lock struts straighten such that the longitudinal segments are disposed father apart form each other.

### FIELD OF THE INVENTION

The invention relates generally to a stent, and in particular, a stent including a toggle lock. The invention has particular application in polymer stents.

### BACKGROUND OF THE INVENTION

Stents have gained acceptance in the medical community as a device capable of supporting body lumens, such as blood vessels, that have become weakened or are susceptible to closure. Typically, a stent is inserted into a vessel of a patient after an angioplasty procedure has been performed to partially open up the blocked/stenosed vessel thus allowing access for stent delivery and deployment. After the catheter used to perform angioplasty has been removed from the patient, a tubular stent, maintained in a small diameter delivery configuration at the distal end of a delivery catheter, is navigated through the vessels to the site of the stenosed area. Once positioned at the site of the stenosis, the stent is released from the delivery catheter and expanded radially to contact the inside surface of the vessel. The expanded stent provides a scaffold-like support structure to maintain the patency of the region of the vessel engaged by the stent, thereby promoting blood flow. Physicians may also elect to deploy a stent directly at the lesion rather than carrying out a pre-dilatation procedure. This approach requires stents that are highly deliverable i.e. have low profile and high flexibility.

These non-surgical interventional procedures often avoid the necessity of major surgical operations. However, one common problem associated with these procedures is the potential release of embolic debris into the bloodstream that can occlude distal vasculature and cause significant health problems to the patient. For example, during deployment of a stent, it is possible for the metal struts of the stent to cut into the stenosis and shear off pieces of plaque which become embolic debris that can travel downstream and lodge somewhere in the patient's vascular system. Further, pieces of plaque material can sometimes dislodge from the stenosis during a balloon angioplasty procedure and become released into the bloodstream.

Various types of endovascular stents have been proposed and used as a means for preventing restenosis. A typical stent is a tubular device capable of maintaining the lumen of the artery open. One example includes the metallic stents that have been designed and permanently implanted in arterial vessels. Metallic stents have low profile combined with high strength. Restenosis has been found to occur, however, in some cases despite the presence of the metallic stent. In addition, some implanted stents have been found to cause undesired local thrombosis. To address this, some patients receive anticoagulant and antiplatelet drugs to prevent local thrombosis or restenosis, however this prolongs the angioplasty treatment and increases its cost.

A number of non-metallic stents have been designed to address the concerns related to the use of permanently implanted metallic stents. U.S. Pat. No. 5,984,963 to Ryan et al., discloses a polymeric stent made from resorbable polymers that degrades over time in the patient. U.S. Pat. No. 5,545,208 to Wolff et al. discloses a polymeric prosthesis for insertion into a lumen to limit restenosis. The prosthesis carries restenosis-limiting drugs that are released as the prosthesis is resorbed. The use of resorbable polymers, however, has drawbacks that have limited the effectiveness of polymeric stents in solving the post-surgical problems associated with balloon angioplasty.

Polymeric stents are typically made from bioresorbable polymers. Materials and processes typically used to produce resorbable stents result in stents with low tensile strengths and low modulus, compared to metallic stents of similar dimensions. The limitations in mechanical strength of the resorbable stents can result in stent recoil after the stent has been inserted. This can lead to a reduction in luminal area and hence blood flow. In severe cases the vessel may completely re-occlude. In order to prevent the recoil, polymeric stents have been designed with thicker struts (which lead to higher profiles) or as composites to improve mechanical properties. The use of relatively thick struts makes polymeric stents stiffer and decreases their tendency to recoil, but a significant portion of the lumen of the artery can be occupied by the stent. This makes stent delivery more difficult and can cause a reduction in the area of flow through the lumen. A larger strut area also increases the level of injury to the vessel wall and this may lead to higher rates of restenosis i.e. re-occlusion of the vessel. Thus, there exists a need for a bioresorbable stent with improved mechanical strength. Similarly, a stent design that improves mechanical strength of the stent can be used with metallic stents to further reduce the profile of the stent.

### BRIEF SUMMARY OF THE INVENTION

The present disclosure relates to stent including a toggle lock connecting segments of the stent together. The stent may be a polymeric stent. The stent includes a plurality of cylindrical rings disposed adjacent to each other. Each cylindrical ring includes a plurality of longitudinal segments coupled to each other by toggle lock struts. The toggle lock struts are disposed substantially at the center of the adjacent longitudinal segments. When the stent is in a compressed state for delivery, the toggle lock struts are bent such that the longitudinal segments are disposed close to each other. Upon expansion of the stent, the toggle lock struts straighten such that the longitudinal segments are disposed father apart form each other. The toggle lock struts “lock” such that they are substantially straight between the longitudinal segments, although the toggle lock struts are curved to follow the circumference of the stent.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of a conventional stent.

FIG. 2 is a side view of a stent in accordance with an embodiment of the present disclosure with the stent in a compressed state for delivery.

FIG. 3 is a side view of the stent of FIG. 1 with the stent in an expanded state.

FIG. 4 is a side view of another embodiment of a stent of the present disclosure with the stent in a compressed state for delivery.

FIG. 5 is a side view of another embodiment of a stent of the present disclosure with the stent in a compressed state for delivery.

FIG. 6 is a perspective view of an embodiment of a toggle lock strut of the present disclosure.

FIG. 7 is a front view of the toggle lock strut of FIG. 6.

FIG. 8 is a perspective view of another embodiment of a toggle lock strut of the present disclosure.

FIG. 9 is a front view of the toggle lock strut of FIG. 8.

FIG. 10 is a perspective view of another embodiment of a toggle lock strut of the present disclosure.

FIG. 11 is a front view of the toggle lock strut of FIG. 10

### DETAILED DESCRIPTION OF THE INVENTION

Specific embodiments of the present disclosure are now described with reference to the figures, wherein like reference numbers indicate identical or functionally similar elements.

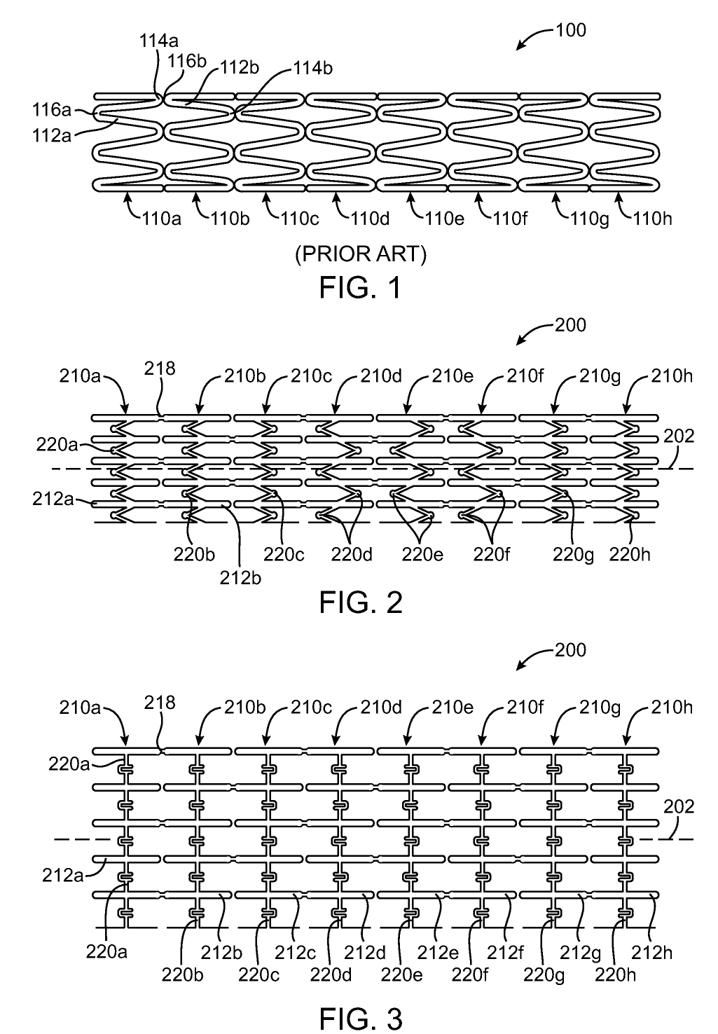

FIG. 1 is a side view of conventional stent 100 known in the art. Stent 100 includes a plurality of cylindrical rings 110a, 110b, 110c, 110d, 110e, 110f, 110g, and 110h disposed adjacent to each other. Each cylindrical ring includes a plurality of substantially straight segments 112 coupled to each other by bends 114, 116. For example, cylindrical ring 110a includes straight segments 112a coupled by bends 114a and 116b. Similarly, cylindrical ring 110b includes straight segments 112b coupled together by bends 114b and 116b. Bends 114 and 116 are peaks and valleys, respectively, of the respective cylindrical ring 110. Cylindrical rings 110 are coupled together at peaks bends 114 and 116. In the particular example shown, a peak bend 114a of cylindrical ring 110a is aligned with a valley bend 116b of adjacent cylindrical ring 110b. Bends 114a and 116b may be coupled to each other by welding or utilizing a connecting element, other ways known to those of ordinary skill in the art. For example, if stent 100 is laser or chemically etched from a tube, bends 114a and 116b may be formed as a unitary piece. Further, in a polymer stent, bends 114a and 116b may be molded as a unitary piece to couple cylindrical rings 110a and 110b together. As would be understood by one of ordinary skill in the art, all of the peak bends 114 of a cylindrical ring 110 may be coupled the adjacent valley bends 116 of an adjacent cylindrical element 110, or only some of peak bends 114 may be coupled to the valley bends 116 of an adjacent cylindrical element 110.

FIG. 2 is a side view of a stent 200 in accordance with an embodiment of the present disclosure. Stent 200 is shown in a compressed state for delivery through a body lumen, although it will be understood by those of ordinary skill in the art that the various features of stent 200 are not drawn to scale. Stent 200 is tubular and includes a longitudinal axis 202. Stent 200 includes a plurality of cylindrical rings 210a, 210b, 210c, 210d, 210e, 210f, 210g, and 210h (generally referred to as cylindrical rings 210). Each cylindrical ring includes a plurality of longitudinal segments 212 coupled to each other by toggle lock struts 220. Toggle lock struts 220 are coupled to longitudinal segments 212 at substantially the longitudinal center of longitudinal segments 212. In the embodiment of FIGS. 2 and 3, longitudinal segments 212 are substantially straight and are substantially parallel to longitudinal axis 202. In the compressed condition shown in FIG. 2, toggle lock struts 220 are bent such that longitudinal segments 212 are close to each other. In such a condition, stent 200 has a smaller diameter for delivery through tortuous passages of the vasculature, for example.

Toggle lock struts 220 may be arranged in several different patterns, as shown in FIG. 2. For example, toggle lock struts 220a and 220b of cylindrical rings 210a and 210b are all bent towards the left side of stent 200. Toggle lock struts 220c, 220g and 220h of cylindrical rings 210c, 210g and 210h are all bent towards the right side of stent 200. Toggle lock struts 220d, 220e and 220f of cylindrical rings 210d, 210e and 210f alternate such that the toggle lock struts within a cylindrical element alternate between being bent to the left and bent to the right of stent 200. Of course, within a stent or within a particular cylindrical ring, various patterns of toggle lock struts can be utilized, as would be apparent to one of ordinary skill in the art.

Cylindrical rings 210 may be coupled to each other using connectors 218. Connectors 218 may unitary pieces with longitudinal segments 212 of adjacent cylindrical rings, as shown. Alternatively, connectors 218 may be welds such that adjacent longitudinal segments 212 are welded to each other. In the embodiment shown in FIG. 2, alternating longitudinal segments 212 of a cylindrical ring 210 are connected to corresponding longitudinal segments 212 of an adjacent cylindrical ring 210. For example, every other longitudinal segment 212a of cylindrical ring 210a is coupled to a corresponding longitudinal segment 212b of cylindrical ring 210b. Such an arrangement improves flexibility over a similar stent with every longitudinal segment 212 coupled to a longitudinal segment 212 of the adjacent cylindrical ring 210. However, one of ordinary skill in the art would recognize that various connecting patterns may be used. For example, only two of the longitudinal segments may be connected to the adjacent cylindrical ring, or every third straight segment, etc. In the embodiment shown in FIG. 2, the connected longitudinal segments 212 are also staggered, such that the connected longitudinal segments 212 between cylindrical rings 210a and 210b are circumferentially offset from the connected longitudinal segments 212 between cylindrical rings 210b and 210c.

FIG. 3 shows stent 220 of FIG. 2 in the expanded state. As can be seen, toggle lock struts 220 have straightened out such that they support the longitudinal segments 212 to which they are connected. As shown in FIG. 3, toggle lock struts 220 are substantially perpendicular to longitudinal axis 202 of stent 200, although one of ordinary skill in the art would recognize that toggle lock struts 220 have some circumferential/radial bend in the direction of arrows 204 because stent 200 is tube-shaped. For the purposes of this invention, the arrows 204 will be referred to as the circumferential direction of stent 200. Thus, in the expanded state, toggle lock struts 220 will generally be parallel to the circumferential direction 204 of stent 200. The term parallel when referring to curved objects/lines refers to generally parallel curved lines, such as railroad tracks.

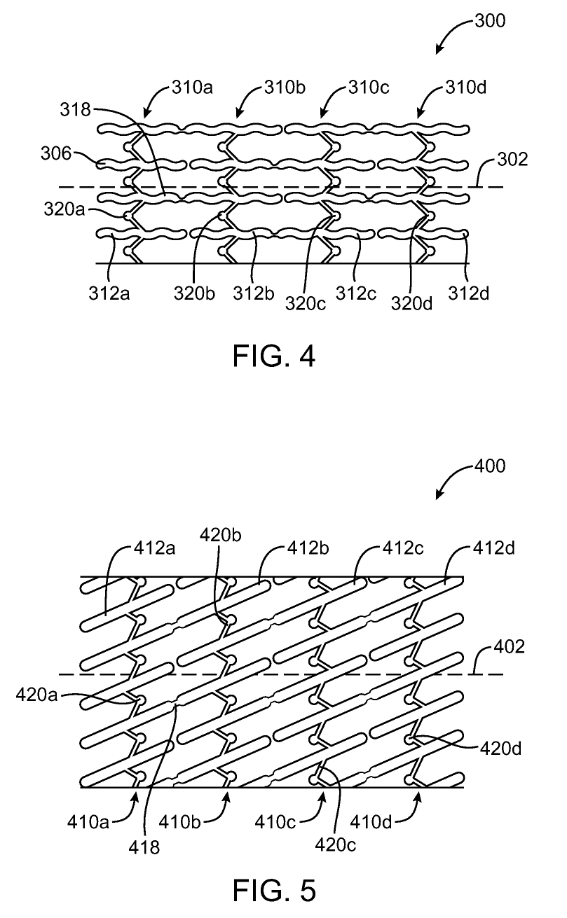

FIG. 4 shows an alternative embodiment of a stent 300 in accordance with the present disclosure. Stent 300 is similar to stent 200 of FIGS. 2 and 3, except that longitudinal segments 312a, 312b, 312c, and 312d of cylindrical rings 310a, 310b, 310c, and 310d are curved. An axis 306 through longitudinal segments 312 is parallel to longitudinal axis 302 of stent 300. As in FIGS. 2 and 3, toggle lock struts 320a, 320b, 320c, and 320d connect longitudinal segments 312a, 312b, 312c, and 312d, respectively, and are connected to generally the longitudinal center of the respective longitudinal segments. FIG. 4 shows stent 300 in the compressed state. Upon expansion, toggle lock struts 320 of stent 300 straighten similar to toggle lock struts 220 as shown in FIG. 3.

FIG. 5 shows an alternative embodiment of a stent 400 in accordance with the present disclosure. Stent 400 is similar to stent 200 of FIGS. 2 and 3, except that longitudinal segments 412a, 412b, 412c, and 412d of cylindrical rings 410a, 410b, 410c, and 410d are disposed at an angle 408 to longitudinal axis 402 of stent 400. As in FIGS. 2 and 3, toggle lock struts 520a, 520b, 520c, and 520d connect longitudinal segments 512a, 512b, 512c, and 512d, respectively, and are connected to generally the longitudinal center of the respective longitudinal segments. FIG. 5 shows stent 400 in the compressed state. Upon expansion, toggle lock struts 420 of stent 400 straighten similar to toggle lock struts 220 as shown in FIG. 3.

As would be understood by one of ordinary skill in the art, several variations of the longitudinal segments may be utilized, and FIGS. 2-5 merely provide some examples. Further, different variations may be used in the same stent. For example, a stent may include some cylindrical rings with longitudinal segments that are straight and parallel to the stent longitudinal axis, as shown in FIGS. 2 and 3, some cylindrical rings that include curved longitudinal segments as shown in FIG. 4, and some cylindrical rings with that are angled as shown in FIG. 5.

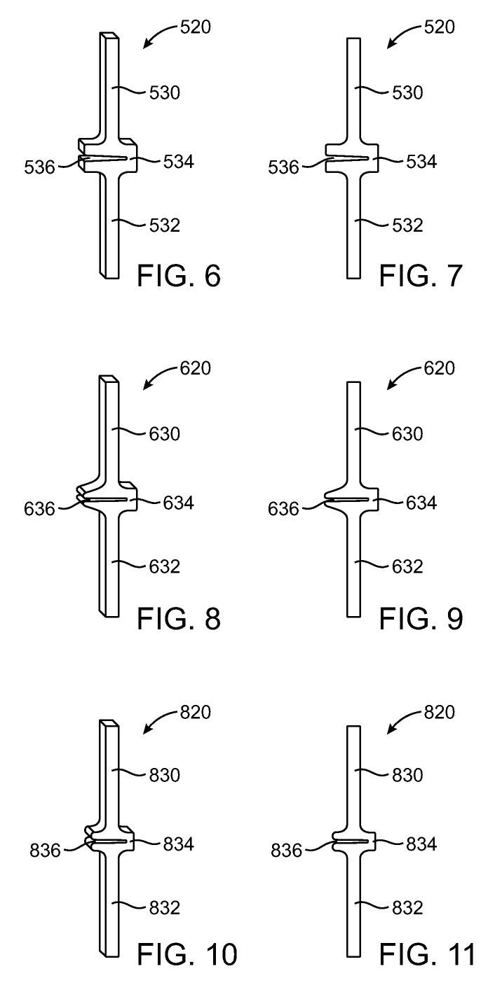

FIGS. 6 and 7 show an embodiment of a toggle lock strut 520. As shown, toggle lock strut 510 includes two arms 530, 532 coupled together at an elbow 534. Elbow 534 includes a slit 536 to enable elbow 534 to bend when a stent is in a compressed condition such as shown in FIGS. 2, 4 and 5. FIGS. 6 and 7 show toggle lock strut 520 when a stent is in the expanded condition, as shown in FIG. 3.

Similarly, FIGS. 8 and 9 show an embodiment of a toggle lock strut 620 including two arms 630, 632 coupled together at an elbow 634. Elbow 634 includes a slit 636 to enable elbow 634 to bend. FIGS. 10 and 11 show an embodiment of a toggle lock strut 820 including two arms 830, 832 coupled together at an elbow 834. Elbow 834 includes a slit 836 to enable elbow 834 to bend. Toggle lock struts 520, 620, and 820 are similar except that the size and/or shape of elbows 534, 634, and 834 vary. As would be understood by one of ordinary skill in the art, various shapes, sizes, and structures can be used for the toggle lock struts.

Stents 200, 300, 400 may be made of materials commonly used for stents, such as stainless steel, “MP35N,” “MP20N,” nickel titanium alloys such as Nitinol, tantalum, platinum-iridium alloy, gold, magnesium, L605, or combinations thereof. “MP35N” and “MP20N” are trade names for alloys of cobalt, nickel, chromium and molybdenum available from standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Stent 200 made of a metallic material may be made in by process known to those of ordinary skill in the art. For example, and not by limitation, a thin-walled, small diameter metallic tube is cut to produce the desired stent pattern, using methods such as laser cutting or chemical etching. The cut stent may then be descaled, polished, cleaned and rinsed. Some examples of methods of forming stents and structures for stents are shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,935,162 to Dang, U.S. Pat. No. 6,090,127 to Globerman, and U.S. Pat. No. 6,730,116 to Wolinsky et al., each of which is incorporated by reference herein in its entirety.

Further, stents 200, 300, 400 may be made of a polymer material suitable for use in a human body. Examples of polymers include but are not limited to, poly-a-hydroxy acid esters such as, polylactic acid (PLLA or DLPLA), polyglycolic acid, polylactic-co-glycolic acid (PLGA), polylactic acid-co-caprolactone; poly(block-ethylene oxide-block-lactide-co-glycolide) polymers (PEO-block-PLGA and PEO-block-PLGA-block-PEO); polyethylene glycol and polyethylene oxide, poly(block-ethylene oxide-block-propylene oxide-block-ethylene oxide); polyvinyl pyrrolidone; polyorthoesters; polysaccharides and polysaccharide derivatives such as polyhyaluronic acid, poly(glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclodextrin sulfobutyl ethers; polypeptides and proteins, such as polylysine, polyglutamic acid, albumin; polyanhydrides; polyhydroxy alkonoates such as polyhydroxy valerate, polyhydroxy butyrate, and the like.

Stents 200, 300, 400 made of a polymeric material may be formed by injection molding, spraying, or casting, or any other methods known to one of ordinary skill in the art.

Stents 20, 300, 4000 can be coated with a therapeutic substance. Further, stents 200, 300, 400 can be formed with recesses or openings in which to deposit such therapeutic substances. Examples of therapeutic substances include, but are not limited to, antineoplastic, antimitotic, antiinflammatory, antiplatelet, anticoagulant, anti fibrin, antithrombin, antiproliferative, antibiotic, antioxidant, and antiallergic substances as well as combinations thereof. Examples of such antineoplastics and/or antimitotics include paclitaxel (e.g., TAXOL® by Bristol-Myers Squibb Co., Stamford, Conn.), docetaxel (e.g., Taxotere® from Aventis S. A., Frankfurt, Germany), methotrexate, azathioprine, vincristine, vinblastine, fluorouracil, doxorubicin hydrochloride (e.g., Adriamycin® from Pharmacia & Upjohn, Peapack N.J.), and mitomycin (e.g., Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax™ (Biogen, Inc., Cambridge, Mass.). Examples of such cytostatic or antiproliferative agents include angiopeptin, angiotensin converting enzyme inhibitors such as captopril (e.g., Capoten® and Capozide® from Bristol-Myers Squibb Co., Stamford, Conn.), cilazapril or lisinopril (e.g., Prinivil® and Prinzide® from Merck & Co., Inc., Whitehouse Station, N.J.), calcium channel blockers (such as nifedipine), colchicine, fibroblast growth factor (FGF) antagonists, fish oil (omega 3-fatty acid), histamine antagonists, lovastatin (an inhibitor of HMG-COA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents that may be used include alpha-interferon, genetically engineered epithelial cells, and dexamethasone. In other examples, the therapeutic substance is a radioactive isotope for implantable device usage in radiotherapeutic procedures. Examples of radioactive isotopes include, but are not limited to, phosphoric acid (H₃P³²O₄), palladium (Pd¹⁰³), cesium (Cs¹³¹), and iodine (I¹²⁵). While the preventative and treatment properties of the foregoing therapeutic substances or agents are well-known to those of ordinary skill in the art, the substances or agents are provided by way of example and are not meant to be limiting. Other therapeutic substances are equally applicable for use with the disclosed methods and compositions.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A stent comprising:
a plurality of cylindrical rings coupled together to form a tube having a longitudinal axis and a circumference, wherein each cylindrical ring includes,
a plurality of longitudinal segments each having a first end and a second end, and
toggle lock struts coupling adjacent segments to each other, wherein the toggle lock struts are coupled to each segment substantially centered between the first end and the second end, wherein the toggle lock struts are bent when the stent is in a compressed state and are substantially parallel to the circumferential direction when in an expanded state.

2. The stent of claim 1, wherein the toggle lock struts comprise a first arm, a second arm, and an elbow coupled to the first arm and the second arm.

3. The stent of claim 1, wherein the longitudinal segments are substantially parallel to the longitudinal axis.

4. The stent of claim 1, wherein the longitudinal segments are substantially straight.

5. The stent of claim 1, wherein the longitudinal segments are curved.

6. The stent of claim 1, wherein the longitudinal segments are disposed at an angle relative to the longitudinal axis such that the longitudinal segments are not parallel to the longitudinal axis.

7. The stent of claim 1, wherein the longitudinal segments are made from a polymeric material.

8. The stent of claim 7, wherein the polymeric material is selected from the group consisting of poly-a-hydroxy acid esters, poly(block-ethylene oxide-block-lactide-co-glycolide) polymers, polyethylene glycol and polyethylene oxide, poly(block-ethylene oxide-block-propylene oxide-block-ethylene oxide), polyvinyl pyrrolidone, polyorthoesters, polysaccharides and polysaccharide derivatives, polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, polypeptides, polyanhydrides, and polyhydroxy alkonoates.

9. The stent of claim 7, wherein the toggle lock struts are made from a polymeric material.

10. The stent of claim 9, wherein the polymeric material is selected from the group consisting of poly-a-hydroxy acid esters, poly(block-ethylene oxide-block-lactide-co-glycolide) polymers, polyethylene glycol and polyethylene oxide, poly(block-ethylene oxide-block-propylene oxide-block-ethylene oxide), polyvinyl pyrrolidone, polyorthoesters, polysaccharides and polysaccharide derivatives, polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, polypeptides, polyanhydrides, and polyhydroxy alkonoates.

11. The stent of claim 1, wherein the toggle lock struts are made from a polymeric material.

12. The stent of claim 1, wherein the longitudinal segments are made from a metal material.

13. The stent of claim 12, wherein the toggle lock struts are made from a metal material.

14. The stent of claim 1, wherein the toggle lock struts are made from a metal material.
